(19) United States
(12) Patent Application Publication
(10) Pub. No.: US 2013/0338767 A1
(43) Pub. Date: Dec. 19, 2013

(54) DEVICES AND METHODS FOR DYNAMIC FOCUSING MOVEMENT

(71) Applicants: Rudy Mazzocchi, Roanoke, VA (US); Ronald David Blum, Roanoke, VA (US)

(72) Inventors: Rudy Mazzocchi, Roanoke, VA (US); Ronald David Blum, Roanoke, VA (US)

(73) Assignee: Elenza Inc.

(21) Appl. No.: 13/976,406

(22) PCT Filed: Dec. 28, 2011
(86) PCT No.: PCT/US11/67516

§ 371 Date: Sep. 3, 2013

Related U.S. Application Data
(60) Provisional application No. 61/428,072, filed on Dec. 29, 2010.

(51) Int. Cl. A61F 2/16

(52) U.S. Cl. CPC A61F 2/1635
USPC 623/6.22

(57) ABSTRACT

An implantable ophthalmic device with one or more optical elements coupled to one or more shape-memory members provides dynamically variable optical power to restore lost accommodation in individuals suffering from presbyopia or aphakia. Running current from a power supply through the shape-memory members causes the shape-memory members to heat up. Once the current heats the shape-memory members past a forward phase-transition temperature, the shape-memory members change shape, which, in turn, causes the optical element(s) to move, yielding a corresponding change in effective optical power. Cooling the shape-memory members (e.g., by reducing or stopping the flow of current) below a reverse phase-transition temperature causes the shape-memory members to return to their original shape, which, in turn, restoring the optical element(s) to their original positions and returning the effective optical power to its original level.

### CROSS-REFERENCE TO RELATED APPLICATIONS

The present application claims the benefit under 35 U.S.C. §119(e) of U.S. Provisional Application No. 61/428,072, filed Dec. 29, 2010, and entitled “Intraocular Lens with Dynamic Focusing Movement,” which is incorporated herein by reference in its entirety.

### BACKGROUND

There are two major conditions that affect an individual's ability to focus on near and intermediate distance objects: presbyopia and pseudophakia. Presbyopia is the loss of accommodation of the crystalline lens of the human eye that often accompanies aging. In a presbyopic individual, this loss of accommodation first results in an inability to focus on near distance objects and later results in an inability to focus on intermediate distance objects. It is estimated that there are approximately 90 million to 100 million presbyopes in the United States. Worldwide, it is estimated that there are approximately 1.6 billion presbyopes.

Tools for correcting presbyopia include reading glasses, multifocal ophthalmic lenses, and contact lenses fit to provide monovision. Reading glasses have a single optical power for correcting near distance focusing problems. A multifocal lens is a lens that has more than one focal length (i.e., optical power) for correcting focusing problems across a range of distances. Multifocal optics are used in eyeglasses, contact lenses, and intra-ocular lenses (IOLs). Multifocal ophthalmic lenses work by means of a division of the lens's area into regions of different optical powers. Multifocal lenses may be comprised of continuous surfaces that create continuous optical power as in a Progressive Addition Lens (PAL). Alternatively, multifocal lenses may be comprised of discontinuous surfaces that create discontinuous optical power as in bifocals or trifocals. A set of contact lenses fit to provide monovision may include two contact lenses, each with a different optical power. One contact lens is for correcting mostly far distance focusing problems and the other contact lens is for correcting mostly near distance focusing problems.

Pseudophakia is the replacement of the crystalline lens of the eye with an IOL, usually following surgical removal of the crystalline lens during cataract surgery. For all practical purposes, an individual will get cataracts if he or she lives long enough. Furthermore, most individuals with cataracts have a cataract operation at some point in their lives. It is estimated that approximately 1.2 million cataract surgeries are performed annually in the United States. In a pseudophakic individual, the absence of the crystalline lens causes a complete loss of accommodation that results in an inability to focus on either near or intermediate distance objects.

Conventional IOLs are monofocal, spherical lenses that provide focused retinal images for far objects (e.g., objects over two meters away). Generally, the focal length (or optical power) of a spherical IOL is chosen based on viewing a far object that subtends a small angle (e.g., about seven degrees) at the fovea. Because a monofocal IOL has a fixed focal length, it cannot mimic or replace the eye's natural accommodation response. Ophthalmic devices with electro-active elements, such as liquid crystal cells, can be used to provide variable optical power as a substitute for the accommodation of an damaged or removed crystalline lens. For example, electro-active elements can be used as shutters that provide dynamically variable optical power as disclosed in U.S. Pat. No. 7,926,940 to Blum et al., which is incorporated herein by reference in its entirety.

### SUMMARY

Embodiments of the present disclosure include an implantable ophthalmic device and associated method of changing the optical power of an implantable ophthalmic device. One exemplary implantable ophthalmic device includes an optical element, a shape-memory member coupled to the optical element, and a power supply, such as a rechargeable battery, operably coupled to the shape-memory member. The power supply may be configured to apply electromagnetic energy, such as a current or voltage, to at least a portion of the shape-memory member so as to cause the shape-memory member to change shape.

In at least one exemplary implantable ophthalmic device with an optical element, the optical element comprises a lens, a prism, an iris, or a spatial light modulator. An illustrative optical element may have a fixed optical power, e.g., about +0 Diopters to about +36 Diopters (e.g., about +0 Diopters, +10 Diopters, +15 Diopters, +20 Diopters, +25 Diopters, +30 Diopters, or any other value from about 0 Diopters to about +36 Diopters). When the implantable ophthalmic device is implanted in an eye, the shape-memory member may move the optical element along an optical axis of the eye when subjected to the electromagnetic energy, e.g., to change the effective optical power of the eye by about 0.1 Diopters to about 3.5 Diopters (e.g., about +0.5 Diopters, +1.0 Diopters, +1.5 Diopters, +2.0 Diopters, +2.5 Diopters, +3.0 Diopters, or any other value from about +0.1 Diopters to about +3.5 Diopters).

Another exemplary implantable ophthalmic device may include a first optical element in optical communication with a second optical element. The first optical element may have a static (fixed) optical power of about −10 Diopters to about +10 Diopters (e.g., about −7.5 Diopters, −5 Diopters, −2.5 Diopters, 0 Diopters, +2.5 Diopters, +5 Diopters, +7.5 Diopters, or any other value from about −10 Diopters to about +10 Diopters) and the second optical element may have a static optical power of about +0 Diopters to about +36 Diopters (e.g., about +5 Diopters, +10 Diopters, +15 Diopters, +20 Diopters, +25 Diopters, +30 Diopters, or any other value from about +10 Diopters to about +36 Diopters). In such an example, when the shape-memory member undergoes a change in shape, the shape-memory member causes the first optical element to move relative to the second optical element, e.g., by about 0.5 mm to about 1.5 mm (0.75 mm, 1.0 mm, 1.25 mm, or any other value from about 0.5 mm to about 1.5 mm). When such a device is implanted in an eye, the relative movement of the first and second optical elements may provide a change in effective optical power of the eye of about +0.5 Diopters to about +3.5 Diopters (e.g., about +1.0 Diopters, +1.5 Diopters, +2.0 Diopters, +2.5 Diopters, +3.0 Diopters, or any other value from about +0.5 Diopters to about +3.5 Diopters).

In one example, the implantable ophthalmic device may include a shape-memory member that comprises a shape-memory alloy, a shape-memory polymer, or both. In another example, the implantable ophthalmic device includes a shape-memory member that comprises a hydrogel. An illustrative shape-memory member may be at least partially coated with an elastic polymeric material, e.g., cross-linked silicone, acrylic co-polymer, polyvinyledene difluoride, polyimide, or any combination thereof. An illustrative shape-memory member can have a thickness of about 0.02 mm to about 0.10 mm and/or a length of about 10.6 mm to about 13.0 mm.

An illustrative shape-memory member may comprise a curved section that straightens when the power supply applies the electromagnetic energy to the shape-memory member. One illustrative shape-memory member changes shape when heated to a temperature of about 35 degrees Celsius to about 45 degrees Celsius (e.g., 36 degrees Celsius, 38 degrees Celsius, 40 degrees Celsius, 42 degrees Celsius, 44 degrees Celsius, or any other value from about 35 degrees Celsius to about 45 degrees Celsius). Causing an illustrative shape-memory member to change shape may involve dissipating a power of about 1.5 microwatts to about 5.0 microwatts (e.g., about 2.0 microwatts, 2.2 microwatts, 2.5 microwatts, 3.0 microwatts, 4.0 microwatts, or any other value of about 1.5 microwatts to about 5.0 microwatts).

Yet another illustrative implantable ophthalmic device can include a first electrode, a second electrode, and a switch. The first and second electrodes are configured to convey current from the power supply to first and second portions, respectively, of the shape-memory member based on actuation of the switch. In one such exemplary implantable ophthalmic device, the first portion of the shape-memory member is configured to change shape independently of the second portion of the shape-memory member. For instance, the switch may be configured to apply current to the first portion of the shape-memory member independent of application of current to the second portion of the shape-memory member. Such a device may also include a third electrode configured to convey current from the power supply to a third portion of the shape-memory member.

Another exemplary implantable ophthalmic device may further comprise a detector configured to provide a trigger signal indicative of the presence of an accommodative trigger; and a processor operably coupled to the detector and the power supply and configured to actuate the power supply in response to the trigger signal.

Embodiments of the present disclosure also include a method of dynamic focusing. An illustrative method comprises applying electromagnetic energy to at least a portion of a shape-memory member anchoring an optical element so as to cause the shape-memory member to move the optical element. Such a method may further include providing a trigger signal representing a presence of an accommodative trigger and applying the electromagnetic energy in response to the trigger signal.

Applying the electromagnetic energy may comprise running a current through the at least a portion of the shape-memory member, e.g., so as to cause part or all of the shape-memory member to undergo a phase transition. Alternatively, or in addition, applying the electromagnetic energy may comprise applying a voltage across the at least a portion of the shape-memory member, e.g., so as to cause part or all the shape-memory member to change shape. Applying the electromagnetic energy to the shape-memory member may cause some or all of the shape-memory member to straighten.

One exemplary method of dynamic focusing may include anchoring the optical element to a structure within the eye, e.g., with an anchor or with the shape-memory member. Applying the electromagnetic energy to the shape-memory member can cause the shape-memory member to move the optical element along an optical axis of the eye. In one case, the optical element is a first optical element, and applying the electromagnetic energy to the shape-memory member causes the shape-memory member to move the first optical element with respect to a second optical element, e.g., by about 0.5 mm to about 1.5 mm, so as to produce a change in effective optical power of the eye, e.g., of about 0.1 Diopters to about 3.5 Diopters. An exemplary method may further include applying electromagnetic energy to another portion of the shape-memory member so as to cause the shape-memory member to move the optical element over an additional distance.

Still another embodiment of the present disclosure includes an implantable ophthalmic device with a first optical element in optical communication with a second optical element. Such an implantable ophthalmic device also includes a shape-memory member coupled to at least one of the first and second optical elements, a power supply operably coupled to the shape-memory member, a detector configured to provide a trigger signal indicative of the presence of an accommodative trigger, and a switch operably coupled to the detector and the power supply. The switch may be configured to apply current from the power supply to at least a portion of the shape-memory member in response to the trigger signal so as to cause the shape-memory member to move the first optical element with respect to the second optical element.

An exemplary implantable ophthalmic device may also include a sensor configured to detect an accommodative stimulus and to provide a signal for triggering a change in optical power in response to the accommodative stimulus. The sensor may provide the signal by detecting a physiological response of an eye, detecting an ambient light level, and generating the signal in response to the ambient light level and to a presence of the physiological response. The sensor may be coupled to a processor that receives the signal and triggers the actuator in response to the signal and a power supply configured to power the electronic components in the implantable ophthalmic device. The components may be encapsulated in a hermetically sealed housing, or capsule, that also encloses the optical element.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

The accompanying drawings, which are incorporated in and constitute a part of this specification, illustrate embodiments of the disclosed technology and together with the description serve to explain principles of the disclosed technology.

FIG. 1 shows a cross section of a healthy human eye.

FIG. 2 is a perspective view of an illustrative implantable ophthalmic device with a single optical element coupled to several shape-memory members.

FIG. 3 illustrates a shape-memory member of FIG. 2 in a curved state (left) and a straightened state (right).

FIGS. 4A and 4B are cross-sectional views of an illustrative implantable ophthalmic device that includes two optical elements coupled together with several shape-memory members implanted in the eye in a first state (FIG. 4A) and a second state (FIG. 4B).

FIGS. 5A and 5B are cross-sectional views of an illustrative implantable ophthalmic device with two optical elements coupled together with segmented shape-memory members in a first state (FIG. 5A) and a second state (FIG. 5B).

FIG. 6 is a plot of Martensite phase transition temperature versus nickel content percentage for nitinol.

FIG. 7 is a plot of strain versus stress for different phases and states of nitinol.

### DETAILED DESCRIPTION

Presently preferred embodiments of the disclosure are illustrated in the drawings. An effort has been made to use the same or like reference numbers to refer to the same or like parts.

### The Eye

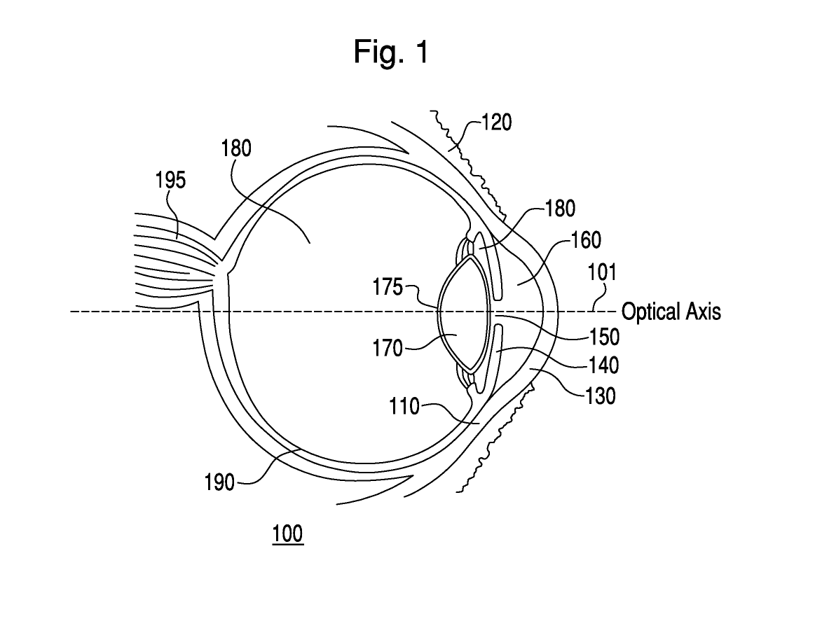

FIG. 1 shows a cross section of a healthy human eye 100 with an optical axis 101 denoted by a dashed line. The white portion of the eye is known as the sclera 110 and is covered with a clear membrane known as the conjunctiva 120. The central, transparent portion of the eye that provides most of the eye's optical power is the cornea 130. The iris 140, which is the pigmented portion of the eye, forms the pupil 150. The sphincter muscles constrict the pupil and the dilator muscles dilate the pupil. The pupil is the natural aperture of the eye. The anterior chamber 160 is the fluid-filled space between the iris and the innermost surface of the cornea. The crystalline lens 170 is held in the lens capsule 175 and provides the remainder of the eye's optical power. The retina 190, which is separated from the back surface of the iris 140 by the posterior chamber 180, acts as the “image plane” of the eye 100 and is connected to the optic nerve 195, which conveys visual information to the brain.

A healthy crystalline lens 170 is capable of changing its optical power such that the eye is capable of imaging objects at near, intermediate, and far distances to the front surface of the retina 190 in a process known as accommodation. Presbyopic individuals suffer from a loss of accommodation, which makes it difficult for them to focus on near objects; as their disease progresses, they eventually lose the ability to focus on intermediate objects as well. An aphakic individual has no crystalline lens and, therefore, cannot focus on object at near or intermediate distances.

Intraocular Lenses and Other Implantable Ophthalmic Devices

An intraocular lens (IOL) may be implanted in the eye of an aphakic individual to replace at least some of the optical power provided by the crystalline lens in a healthy eye. A static IOL, which is generally implanted after cataract extraction, generally cannot provide uncompromised vision at all distances. Mono-focal static IOLs are designed and selected to provide excellent vision at optical infinity, but must be used with reading glasses or bifocal spectacles for uncompromised near and intermediate vision. A multifocal static IOL provides good vision at far and near distances, but produces double images on the retina at all object distances, leading to loss of contrast. It may also cause sensations of ghosting, double images, flare, and glare, all due to the fact that part of the multifocal optic is designed to provide best focus at far distance and another part is designed to provide best focus at near distance.

A passive accommodative IOL is supposed to mimic the behavior of the crystalline lens. Such a passive accommodative IOL includes a flexible lens whose optical power changes when the lens is squeezed circumferentially, just like the crystalline lens. When implanted, capsular contraction and dilation are supposed to change the optical power of the passive accommodative IOL.

An active accommodative IOL includes an actuator that changes the optical power or position of an optical element in response to a signal from a sensor. When the sensor detects an accommodative stimulus, it sends a signal to the actuator, which responds to the signal by changing the size or shape of an electro-active aperture or moving a lens along the eye's optical axis to change the accommodative IOL's effective optical power or depth of field. The actuator changes the aperture back to its original size and shape or moves the lens back to its original position when the need for an accommodative response ends.

Embodiments of the present disclosure include an implantable ophthalmic device that includes an optical element, such as a lens or prism, that is coupled to one or more shape-memory members. In at least one example, the shape-memory members change from a first or relaxed shape, such as a curved shape, to a second or actuated shape, such as a straightened shape, in response to a current or voltage from a power supply, such as a rechargeable battery. In some examples, removing the electrical current or potential from a shape-memory member may cause the shape-memory member to change from the second state to the first state. In other examples, one or more of the shape-memory members may be “bistable”—that is, they may remain in the second shape until they are subject to another current or voltage from the power supply. When the implantable ophthalmic device is implanted in an eye, changing the shapes of the shape-memory members may cause the optical element to translate along the optical axis of the eye so as to change the eye's point of focus, e.g., from a near point to an intermediate point, or from an intermediate point to a far point.

Single-Lens Implantable Ophthalmic Devices

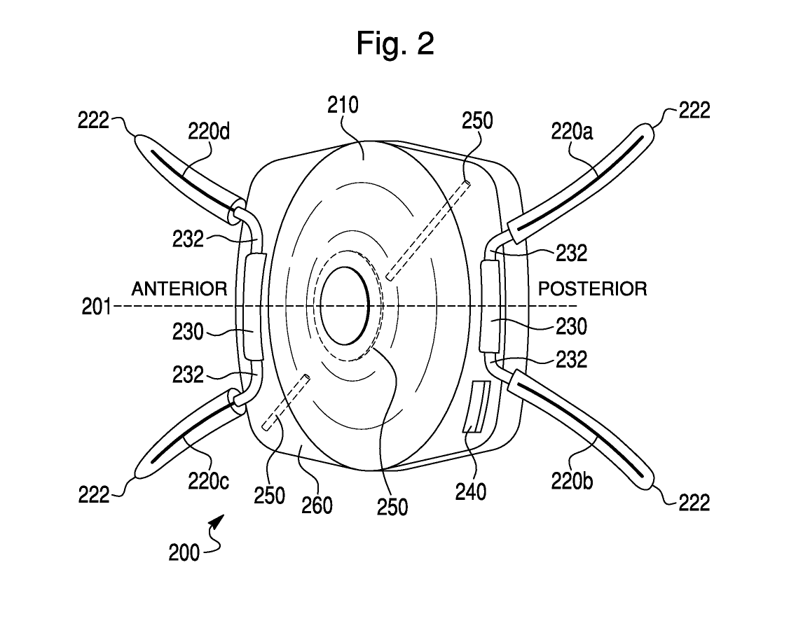

FIG. 2 is a perspective view of an illustrative implantable ophthalmic device 200 with a single optical element 210 according to one embodiment of the present disclosure. Such an illustrative implantable ophthalmic device 200 may be useful for far and intermediate vision, with functional near-vision performance that may be augmented with reading glasses. The implantable ophthalmic device may have a length of about 10.6 mm to about 13.0 mm (e.g., about 11.0 mm to about 12.0 mm) and can be implanted within the eye using any suitable technique.

The optical element 210, shown here as a biconvex lens, that is encased in a hermetically sealed housing 260. Those of skill in the art will readily appreciate that the optical element 210 may also or alternatively include a convex-concave lens, a biconcave lens, a plano-convex lens, plano-concave lens, spherical surface, aspheric surface, a prism, an optical flat, or an electro-active device (e.g., a liquid-crystal spatial light modulator). The optical element 210 has a diameter of about 4.0 mm to about 7.0 mm (e.g., about 4.5 mm, 5.0 mm, 5.5. mm, or 6.0 mm) and a fixed optical power of anywhere from about 0 Diopters to about 36 Diopters.

The implantable ophthalmic device 200 also includes four shape-memory members 220a-220d (collectively, shape-memory members 220) that extend out from the hermetically sealed housing 260. Each shape-memory member 220 may have a thickness of about 0.10 mm to about 0.35 mm (e.g., about 0.10 mm to about 0.20 mm) and a length of about 2.5 mm to about 3.5 mm (e.g., about 3.1 mm to about 3.3 mm). An exemplary shape-memory member 220 may comprise a shape-memory alloy (e.g., nitinol), shape-memory polymer (e.g., polyurethane), a hydrogel, or any other suitable shape-memory material.

The geometry of each shape-memory member 220 can be computed through finite element analysis so as to promote secure engagement with the capsular equator when the device 200 is implanted. Some exemplary shape-memory members 220 may include protrusions, anchors, or be otherwise configured to anchor the implantable ophthalmic device 200 within an eye, whereas other exemplary shape-memory members 220 may be coupled to distinct anchors or protrusions configured to anchor the implantable ophthalmic device 200 within an eye. For instance, a shape-memory member 220 may include or be coupled to a tab that engages a physiological structure within or near the eye.

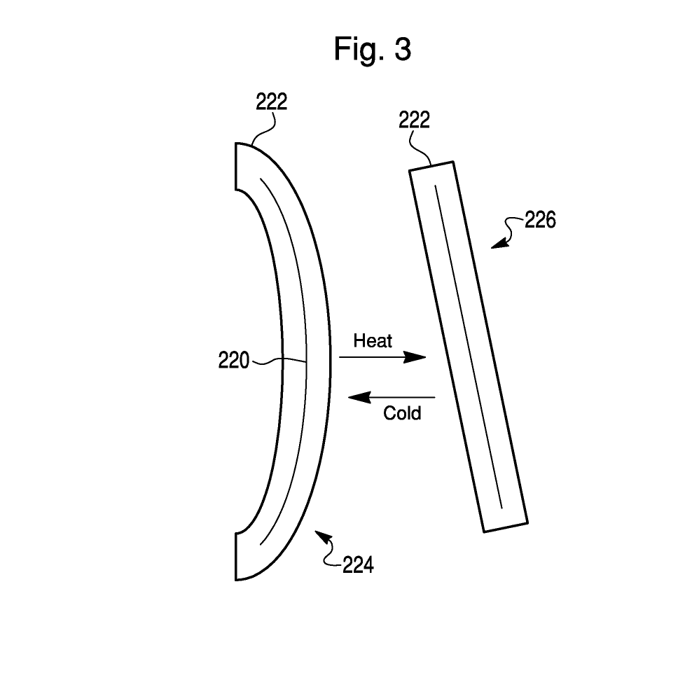

Each shape-memory member 220 has at least two states: a bent or curved state 224 and a linear or straightened state 226, as shown in FIG. 3. Upon being straightened to the straightened state 226, the shape-memory member is preferably linear or substantially linear, however, it also could be merely in a less bent or curved configuration than in the bent or curved state 226. Each shape-memory member 220 may be coated with a biocompatible, cross-linked polymer 222, such as a silicone, acrylic co-polymer, polyimide, fluorocarbon (e.g., polyvinyledene difluoride), or any other suitable material.

Heating the shape-memory member 220 above a forward phase transition temperature causes the shape-memory member 220 to transition from the curved state 224 to the straightened state 226. Cooling the shape-memory member 220 below a reverse phase transition temperature (e.g., by allowing the shape-memory member 220 to return to body temperature) causes the shape-memory member 220 to transition from the curved state 224 to the straightened state 226. In some cases, the forward and reverse phase transition temperatures may be the same; in other cases, they may be different.

The implantable ophthalmic device 200 also includes a power supply 230, a processor 240, and a sensor 250, any or all of which may be enclosed in the hermetically sealed housing 260. The sensor 250, which may include one or more sensing elements, detects an accommodative stimulus indicating the presence of an accommodative response, e.g., by performing a differential measurement of the ambient light level and the size of the pupil 150 (FIG. 1) as described below. The sensor 250 sends a signal representing the presence (or absence) of an accommodative stimulus to the processor 230, which interprets the signal, e.g., by comparing the signal from the sensor 250 to a value stored in memory (not shown). For instance, the processor 250 may search for a value representing the amplitude or timing of the signal in a look-up table stored in an electrically erasable programmable read-only memory (EEPROM).

If the processor 250 determines that the sensor 250 has detected an accommodative stimulus, it applies voltage from the power supply 230 to one or more shape-memory members 220 via one or more electrodes 232 in electrical communication with the power supply 230 and shape-memory members 220. Current flows from the power supply 230 through the shape-memory members 220, causing the shape-memory members to heat up through resistive heating. In some cases, the electrical power dissipated by heating each shape-memory member 220 ranges from about 1.5 microwatts to about 5.0 microwatts (e.g., about 2.2 microwatts). (In other embodiments, the shape-memory member may change shape upon application of an electric voltage.) An exemplary power supply 230 may provide up to 25 microamp-hours per day and, optionally, may be able to provide about three to about ten days (e.g., about six days) of operation (e.g., about 150 microamp-hours) before requiring recharging.

The resistive heating causes the temperature of each shape-memory member 220 to rise above its phase transition temperature, which, in turn, causes the shape-memory member 220 to transition from the curved shape 224 to the straightened shape 226 in which the shape-memory member 220 forms an angle (e.g., of about 24°) with the optical axis 101 of the eye 100. When the implantable ophthalmic device 200 is implanted properly in the eye 100 (FIG. 1), the shape-memory members' change in shape may cause the optical element 210 to move in an anterior direction (i.e., toward the cornea 130 and away from the optic nerve 195). When the accommodative stimulus is no longer present, the processor 250 may stop the flow of current from the power supply 230 to the shape-memory members 220, which stops the heating of the shape-memory members 220. Cooling the shape-memory members 220 (e.g., by stopping the flow of current from the power supply 230) causes the optical element 210 to move in a posterior direction (i.e., away from the cornea 130 and toward from the optic nerve 195).

In one example, the optical element 210 may translate along the optical axis 101 of the eye 100 by up to about 1.5 mm, which may cause in an increase of up to about 1.84 Diopters in the eye's effective optical power assuming a refractive index of 1.45. Implanting the implantable ophthalmic device 200 such that the optical element 210 is vaulted posteriorly may further increase the translation distance, e.g., to about 2.0 mm, which corresponds to a change in effective optical power of about 2.5 Diopters. Longer or shorter shape-memory members 210 may be capable of moving the optical element by larger or smaller distances, although care should be taken to prevent uncontrolled movement of the optical element 210 toward the iris 140 or the corneal endothelium and to prevent the iris 140 from capturing or trapping the optical element 210.

Multi-Optic Implantable Ophthalmic Devices

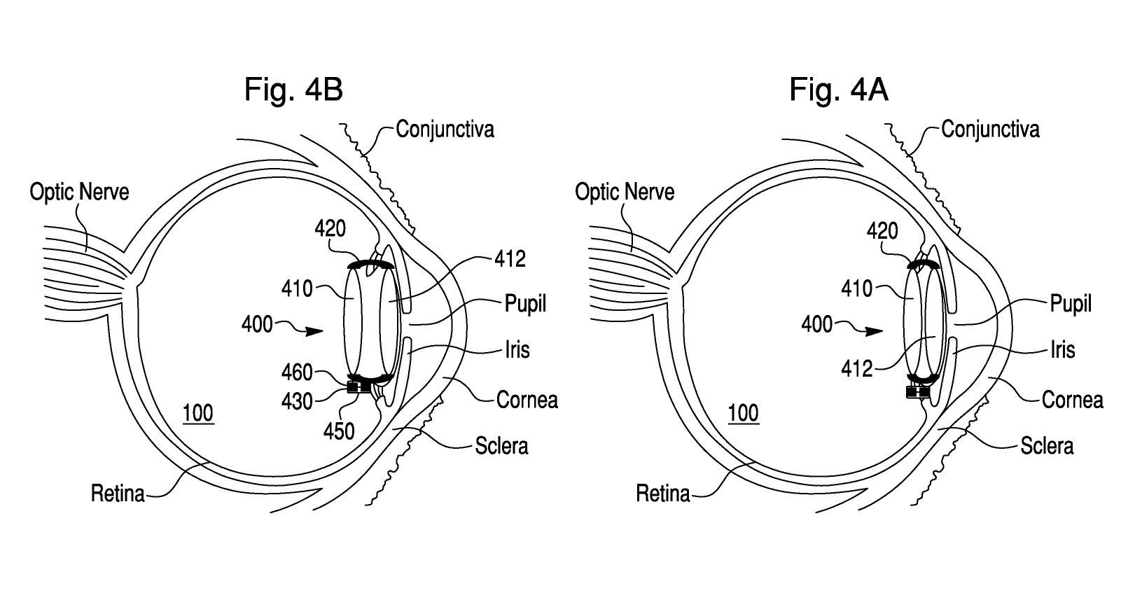

FIGS. 4A and 4B show an illustrative double-optic implantable ophthalmic device 400 that may be suitable for viewing distant and near objects in a low optical power state (left) and a high optical power state (right). The implantable ophthalmic device 400 may also provide functional intermediate vision, e.g., by enhancing the depth of focus of the eye 100 by adding asphericity to one or both of the optical elements 410 and 412, which are depicted as biconvex lenses. Those of skill in the art will readily appreciate that other arrangements of optical elements 410 and 412 are also possible; for instance, optical element 410 may be a biconvex (positive) lens and optical element 412 may be a convex-concave (negative) lens or a biconcave (negative) lens. In general, optical element 410 may have a static optical power of about −10 Diopters to about +10 Diopters, and optical element 412 may have a static optical power of about 0 Diopters to about +36 Diopters. Alternatively, or in addition, one or both optical elements 410 and 412 may provide a dynamic or a variable aperture through which light passes.

The optical elements 410 and 412 are coupled to and spaced apart from each other by one or more shape-memory members 420, each of which may comprise nitinol or another suitable shape-memory material. Each shape-memory member 420 is coated with a cross-linked polymer 422 and changes shape (e.g., from a curved shape to a linear shape as shown in FIG. 3) as it undergoes a phase transition induced by application of electromagnetic energy from a power supply 430, which is enclosed in a hermetically sealed cavity 460. The hermetically sealed cavity 460 also encloses a processor 440 and a sensor 450, both of which are electrically coupled to the power supply 430.

In operation, the sensor 450 monitors changes in the eye 100 and/or the surrounding environment (e.g., the ambient light level) for an accommodative stimulus. The sensor 450 transmits a signal to the processor 440, which determines whether or not an accommodative stimulus is present based on at least one attribute (e.g., the amplitude) of the signal. If the processor 440 determines that an accommodative stimulus is present, it puts one or more of the shape-memory members 420 in electrical communication with the power supply 430. As a result, current runs from the power supply 430 through the shape-memory members 420. The current induces resistive heating in the shape-memory members 420.

In at least one embodiment, heating the shape-memory members 420 above a phase transition temperature, which may be from about 35° C. to about 45° C., causes the shape-memory members 420 to change shape as described above. Because the shape-memory members 420 are coupled to the optical elements 410 and 412, the change in shape results in movement of the optical elements 410 and 412 with respect to each other. For instance, optical element 412 may be anchored to a physiological structure within the eye 100 using an anchor (not shown) or even one or more of the shape-memory members 420, and optical element 410 may move as the shape-memory members 420 change shape, as shown in FIGS. 4A and 4B, or vice versa. Alternatively, both optical elements 410 and 412 may move relative to the eye 100 when one or more of the shape-memory members 420 change shape.

Depending on the exact configuration, the optical elements 410 and 412 may move relative to each other over a range of about 0.5 mm to about 1.5 along the optical axis 101 (FIG. 1) of the eye 100. Depending on the optical powers of the optical elements 410 and 412 and the distance(s) between the optical elements 410 and 412, such relative motion may produce a change in the effective optical power of the eye of about 3.0 Diopters/mm, or about 1.5 Diopters to about 4.5 Diopters total. Those of skill in the art will readily appreciate that other ranges of motion and changes of effective optical power are also possible.

Implantable Ophthalmic Devices with Segmented Shape-Memory Members

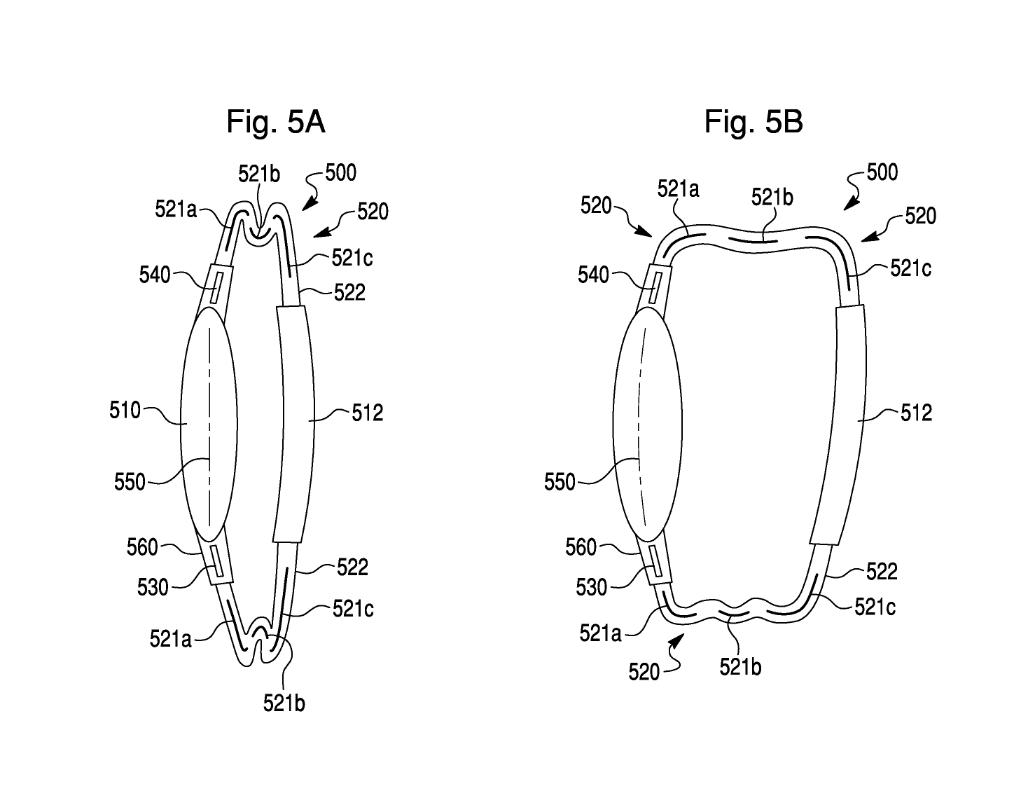

FIGS. 5A and 5A show an exemplary implantable ophthalmic device 500 that includes optical elements 510 and 512 coupled together by one or more segmented shape-memory members 520. In this case, optical element 510 is depicted as a biconvex lens and optical element 512 is depicted is a convex-concave lens, but those of skill in the art will readily appreciate that other types and arrangements of optical elements are possible as well. Optical element 510 may have a static optical power of anywhere from about 0 Diopters to about +36 Diopters, and optical element 512 may have a static optical power of anywhere from about −10 Diopters to about 0 Diopters. Optical element 510 is encapsulated in a hermetically sealed cavity 560 along with a power supply 530, processor 540, and sensor 550 that are in electrical communication with each other and that may operate in a fashion similar to the power supplies, processors, and sensors described with respect to FIGS. 2, 4A, and 4B.

One or more of the shape-memory members 520 includes three segments 521a-521b (collectively, segments 521) that are encased in cross-linked polymer 522 or another suitable material. Other embodiments may include two, four, five, or more segments 521 per shape-memory member 520, and different shape-memory members 520 in the same implantable ophthalmic device 500 may have different numbers, shapes, or arrangements of segments 521. For instance, example, each segment 521 may be a different piece of nitinol wire or ribbon that is curved or angled and has a total length of about 0.5 mm to about 1.5 mm (e.g., about 0.6 mm, 0.7 mm, 0.8 mm, 0.9 mm, 1.0 mm, 1.1 mm, or 1.2 mm). The segments 521 comprising a single shape-memory member 520 may be electrically isolated each other.

Each segment 521 is coupled to the power supply 530 via a respective set of electrodes (omitted for clarity). Running a current from the power supply 530 through a given segment 521 or group of segments 521 induces resistive heating of the given segment 521 or group of segments 521, causing the given segment 521 or group of segments 521 to change shape as described above (e.g., with respect to FIG. 3). Because each segment 521 is coupled to its own set of electrodes, the processor 540 can actuate each segment 521 independently of the other segments 521 in the shape-memory member 520. As a result, the processor 540 can effect stepped changes in effective optical power by actuating one, some, or all of the segments 521 in a given shape-memory member 520. For instance, FIGS. 5A and 5B illustrate the implantable ophthalmic device 500 with all segments 521 in a curved (low-temperature) state (left) and segments 521b in a linear (high-temperature) state.

Segmented shape-memory members 520 may be used to provide a finer degree of control of the change in effective optical power provided by the implantable ophthalmic device 200. The segments 521 may provided evenly spaced changes in optical power (e.g., changes of about 0.5 Diopters to about 1.2 Diopters each) or different change in optical power (e.g., changes of about 0.75 Diopters, about 1.25 Diopters, and about 1.5 Diopters) as desired. Actuating (heating) all of the segments 521 may provide full accommodation for viewing objects at near distances (e.g., less than 1 m), whereas actuating (heating) only some of the segments 521 may provide partial accommodation for viewing objects at intermediate distances (e.g., about 1 m to about 5 m). Leaving the implantable ophthalmic device 200 in its low-temperature (unactuated) state may provide vision at for distant objects (e.g., objects over about 5 m away).

Nitinol Shape-Memory Members

Suitable shape-memory members may be formed of a nitinol wire having a length of about 2.5 mm to about 3.5 mm (e.g., about 3.1 mm to about 3.3 mm) and a diameter of about 20 microns to about 125 microns (e.g., about 100 microns), or about 3.0 micrograms to about 15.0 micrograms (e.g., about 7.4 micrograms) of nitinol. Nitinol is a biocompatible, nonthrombogenic alloy that can be manufactured according to any suitable standard, including the ASTM F2063-00 “Standard Specification for Wrought Nickel-Titanium Shape Memory Alloys for Medical Devices and Surgical Implants.” Nitinol has a resistivity of about 76 μΩ-cm in its low-temperature state and about 82 μΩ-cm in its high-temperature state. It has a thermal conductivity 0.1 W/cm ° C., a heat capacity of 0.077 cal/gm ° C., and a latent heat of 5.78 cal/gm (24.2 J/gm). Nitinol has an ultimate tensile strength of about 754 MPa to about 960 MPa (about 110 ksi to about 140 ksi), a typical elongation to fracture of about 15.5%, a high-temperature typical yield strength of about 560 MPa (80 ksi), a low-temperature typical yield strength 100 MPa (15 ksi), a high-temperature elastic modulus of about 75 GPa (11 Mpsi), and a low-temperature elastic modulus 28 GPa (4 Mpsi).

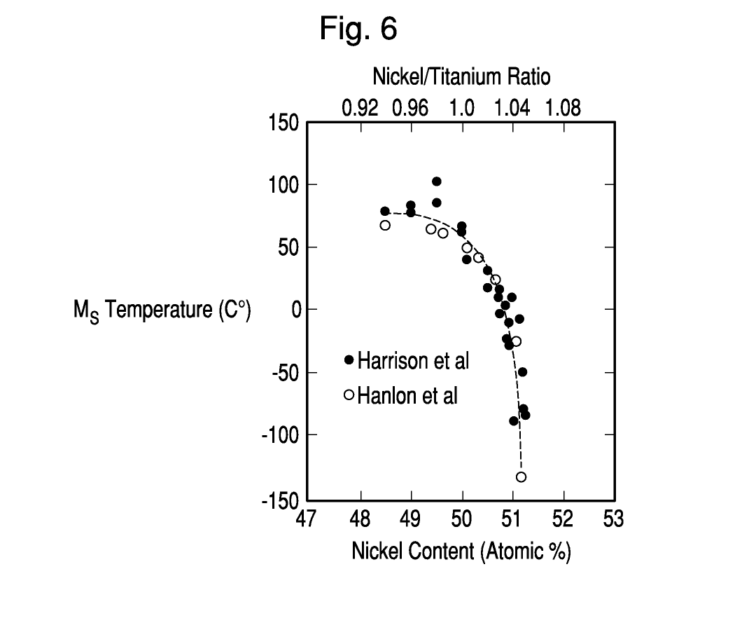

Nitinol undergoes a transition from a Martensite phase to an Austenite phase at a temperature M_{f}. Nitinol also undergoes a transition from the Austenite phase to the Martensite phase at a temperature Mₛ, which may be lower that the temperature M_{f}. The exact phase transition temperatures can be chosen to be anywhere from about −100° C. to about 100° C. by appropriately choosing the nitinol nickel content as shown in FIG. 6, which is a plot of Mₛ versus nickel content. In some examples, the phase transition temperatures M_{f} and Mₛ may be selected to be slightly higher than body temperature (e.g., between about 35° C. and about 45° C.) by choosing a nitinol alloy that comprises nickel at a mole percent range of about 50.1% to about 53.0%.

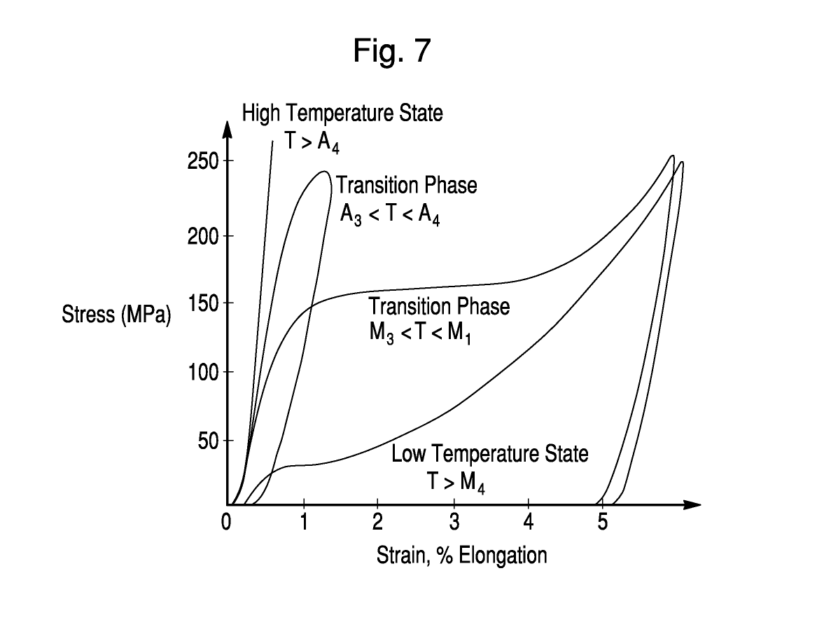

Heating nitinol above its Martensite-to-Austenite phase transition temperature causes the nitinol to undergo a phase transition in which the nitinol changes shape, but not volume (or density) thanks to a twinning atomic transition, as shown in FIG. 7, which is a plot of stress versus strain from nitinol in different states and phases. Nitinol in its Martensite state can accept up to 10% strain without breaking and has a demonstrated fatigue resistance up to 10 million cycles, when strain-induced cycling may occur. For instance, a 100-micron thick nitinol wire embedded in a polymeric coating may produce about 1 lb of force when heated or cooled past a phase transition temperature.

Power Supplies

As noted above, a power supply (e.g., power supplies 230, 430, or 530) provides a voltage or current to change the state of the shape-memory members and may also provide electrical power to electronic components, such as a processor or detector, in the implantable ophthalmic device. In at least one example, the power supply includes a solar cell, capacitor, or thin-film rechargeable battery like those manufactured by Excellatron, Wyon, or Front Edge. Suitable power sources include rechargeable lithium-ion batteries with a minimum of 1,000 cycles of recharging, a diameter in the range about 0.8 mm to about 2.5 mm (e.g., about 0.8 mm to about 1.3 mm), and a thickness of about 500 microns to about 3.0 mm (e.g., about 0.7 mm to about 2.0 mm). If desired, the batteries may be recharged using an inductive antenna as described in PCT/US2011/040896 filed Jun. 17, 2011, and entitled, “ASIC Design and Function,” and PCT/US2011/050533 filed Sep. 6, 2011, and entitled, “Installation and Sealing of a Battery on a Thin Glass Wafer to Supply Power to an Intraocular Implant,” each of which is incorporated herein by reference in its entirety.

Thin-film rechargeable batteries are particularly well-suited for use in implantable ophthalmic devices because they can be cycled more 45,000 times, which could translate to a usable lifetime of 20-25 years in the lens or optic. Two thin film rechargeable batteries may be used and may stacked one atop the other. In this configuration, one of the batteries may be used for 20-25 years and the other battery may be switched to when the first battery is no longer operable. Alternatively, the other battery may be switched to by a signal sent remotely to the controller. This may extend the lifetime of the optic or lens to 40-50 years.

One or more light-sensitive cells, such as solar cells or photovoltaic cells, may also be used to supplement, augment, and/or obviate the need for a battery. The light-sensitive cell is located out of the user's line of sight of the user, e.g., peripheral to the margin of the pupil when partially dilated by darkness, but not fully dilated. The device may thus be charged by using an eye-safe laser capable of energizing the light-sensitive cell or cells.

Alternatively, the light-sensitive cell may be located in front of (closer to the cornea of the eye) and separately disposed from a portion of the iris of a user's eye. Thin electrical wiring may operably connect the solar cell to the controllers. The electrical wiring may pass through the pupil without touching the iris and operably connect to the implantable ophthalmic device. The solar cell may be large enough such that it supplies enough electrical power to obviate the need for a separate power supply. The thin electrical wiring may not conduct electricity and may have a form factor which has the appropriate tensile strength to hold the solar cell in place. In some configurations, one or more small holes may be made in the iris by an ophthalmic laser such that the thin electrical wiring connects the solar cell to the implantable ophthalmic device.

Programmable Controller(s)

As noted above, an exemplary implantable ophthalmic device may include a controlling system with one or more elements, including at least one programmable controller or processor (e.g., a processor 240, 440, or 540) that receives and processes signals from one or more sensors to determine when the patient is viewing an near or intermediate object. In other words, the controlling system monitors and responds to indications accommodative stimuli. The controller may also receive energy form an antenna and transform the received energy into a voltage suitable for re-charging the power supply. It may also step up or step down a voltage from the power supply (e.g., 1.6 Volts from a lithium-ion battery) into a voltage suitable for providing efficient resistive heating when applied to nitinol or other shape-memory materials as described above. In addition, the controller may also maintain a memory, or data register, that records sensor data and stores instructions for processing the sensor data. The controller may also update the memory and provide an uplink via the antenna to a host computer.

In some embodiments, the controller may include one or more application-specific integrated circuits (ASICs) as disclosed in PCT/US2011/040896 filed Jun. 17, 2011, and entitled, “ASIC Design and Function,” which is incorporated herein by reference in its entirety. The first ASIC, which operates at relatively low voltage, e.g., about 4 V, provides functions such as data storage (memory), battery charging, etc. The second ASIC, which operates at relatively high voltage, e.g., 5-11 V, includes a charge pump that steps up the voltage from a power supply, such as a 1.4 V lithium-ion battery, to the 5-11 V actuation voltage of an electro-active cell. Because most of the electronics operate at low voltage, they consume less power, which increases the useful battery life (and the useful life of the device itself), e.g., to about twenty years or more. In addition, charge pumps consume less power and require less area (i.e., they have smaller footprints) than other DC-DC power converters, which makes it possible to reduce the size and power consumption of the second ASIC. Charge pumps also do not require the expensive inductors or additional semiconductors used in other DC-DC converters.

In some exemplary devices, the functions (and associated functional components) are partitioned among the first (low-voltage) ASIC and second (high-voltage) ASIC as follows. The first ASIC includes the functional blocks that are powered by a radio-frequency (rf) field, including an rf communication section (including an antenna), parts of the power management, and the battery charging. The second ASIC includes the functional blocks that are associated with therapy (variation in optical power). These therapy functional blocks may be powered by one or more batteries. The first and second ASICs communicate via a serial communication interface, which may be housed on the second ASIC and powered through the first ASIC.

The first ASIC regulates the second ASIC. In other words, the first ASIC controls the second ASIC's operational state by initiating “wake-up,” i.e., by causing the second ASIC to transition from an idle (sleep) state in which the second ASIC does not actuate or power the electro-active element or consume much power to an operational state in which the second ASIC steps up the battery voltage and/or actuates or powers the electro-active element. By controlling the operating state of the second (high-voltage) ASIC with the first (low-voltage) ASIC, the ophthalmic device consumes less power than other ophthalmic devices that offer similar functionality to the patient.

The second ASIC may also include a battery voltage level monitor which samples the battery voltage in a periodic fashion while the second ASIC is in both the idle and operational states. When the battery level monitor senses that the battery voltage has dropped below a predetermined threshold, e.g., due to self-discharge, a switch (e.g., a latch element, such as an R-S flip-flop) in the second ASIC opens, disconnecting the second ASIC from the battery to stop further discharge of the battery. Other features for reducing current consumption (and extending the device lifetime) include operating the ASICs at a low clock frequency, making as few gate state transitions as possible, and intermittently enabling analog functional sections whenever possible.

Sensors to Detect Accommodative Stimuli

As described above and shown in FIG. 2, an illustrative implantable ophthalmic device may include one or more sensors (e.g., sensors 250, 450, and 550) that produce electromagnetic or electrochemical signals in response to accommodative stimuli (defined below). In one embodiment, the sensor includes at least two silicon photocells comprising a layer of polycrystalline or amorphous silicon deposited on the inner surface of a substrate or other element using to make an exemplary implantable ophthalmic device. The photocells may be about 0.05 mm by 0.05 mm or a 50-micron circle, ranging from about 0.02 mm by about 0.02 mm to about 1.0 mm by about 1.0 mm. Alternatively, the sensor(s) may include one or more piezoelectric elements that can sense constriction and dilation of ciliary processes. Still other sensors may include one or more motion sensors or accelerometers that detect motions of the eye. These motions can be analyzed by the controller to determine incidences of convergence. One or more sensors may be deployed to enhance accuracy of deployment of the variable optical power provided by an exemplary implantable ophthalmic device.

In some inventive implantable ophthalmic devices, the sensor system includes at least two sensors for distinguishing accommodative stimuli from changes in ambient lights levels and task-induced changes in the pupil diameter. When implanted, the first sensor is disposed completely within the pupil; even when fully constricted, the pupil does not occlude the first sensor, allowing the sensor to make precise measurements of ambient luminous flux levels. The second sensor is disposed, when implanted, such that the pupil occludes part of the second sensor's active area(s) as the pupil dilates and constricts. As a result, the second sensor measures both ambient luminous flux and pupil diameter. A processor estimates the pupil diameter from the measurements and determines whether the pupil is changing in diameter in response to accommodative stimuli or other factors by comparing the estimated pupil diameter and measured ambient light levels to predetermined values. The sensor system sends a signal to an optical component, which in turn can respond by changing optical power to focus for near vision upon detection of accommodative stimuli. Further details of suitable sensors can be found in U.S. Patent Application Publication No. 2010/0004741 filed Jul. 2, 2009, and entitled, “Sensor for Detecting Accommodative Trigger,” and in PCT/US2011/051198 filed Sep. 12, 2011, and entitled, “Method and Apparatus for Detecting Accommodations,” both of which are incorporated herein by reference.

### Hermetically Sealed Cavities

Illustrative implantable ophthalmic devices, including those shown in FIGS. 2 and 3, may include one or more components that are hermetically sealed in a glass, coated plastic, or other highly impermeable material. For instance, an exemplary implantable ophthalmic device may include a hermetically sealed cavity (e.g., cavities 260, 460, and 560) than contains an optical element, a power supply, a logic controller (processor), an antenna, or any combination thereof. An illustrative hermetically sealed cavity may be formed of two or more glass plates, each of which has a thickness of about 25 microns to about 250 microns (e.g., about 25 microns to about 125 microns). The hermetically sealed cavity may be filled with saline or some other suitable fluid in order to minimize reflections or loss of image quality resulting from internal reflections off one or more surfaces of an illustrative implantable ophthalmic device, including surfaces of the various electronic and optical components that form parts of the illustrative implantable ophthalmic device. Once filled with fluid (if desired), the hermetically sealed cavity can be sealed using laser fusion, laser welding, or any other suitable hermetic sealing process. The material forming an illustrative hermetically sealed cavity may be further encapsulated by a transparent, hydrophobic acrylic optical material that is biocompatible and foldable. When completely sealed, an illustrative hermetically sealed cavity can be about 3.5 mm to about 6.5 mm in length, about 3.0 mm to about 6.5 mm in width, and about 1.0 mm to 3.5 mm in thickness.

Alternatively, the illustrative hermetically sealed cavity may be formed by one or more pieces of glass- or SiOₓ-coated plastic, such as acrylic, polyimide, PMMA, PVDF, or any other suitable polymer or fluorocarbon. In some examples, the glass or SiOₓ coating is about 200 nm thick, and the plastic is about 5 microns thick to about 100 microns thick. The plastic is highly impermeable, as highly and moderately permeable plastics swell during use through absorption of moisture. (Some permeable plastics may absorb up to 5-6% moisture.) If the capsule absorbs too much moisture, it will swell enough to crack the coating.

Inventive implantable ophthalmic devices may take the form of IOLs, intraocular optics (IOOs), corneal inlays, and corneal onlays. An inventive implantable ophthalmic device may be inserted or implanted in the anterior chamber or posterior chamber of the eye, into the capsular sac, or the stroma of the cornea (similar to a corneal inlay), or into the epithelial layer of the cornea (similar to a corneal onlay), or within any anatomical structure of the eye. An inventive implantable ophthalmic device may have one or more thin, hinge-like sections that allow the implantable ophthalmic device to be folded before implantation and unfolded once positioned properly in a patient's eye. When implanted, partially transparent and opaque elements, such as the sensor, processor, and battery, may be disposed out of the patient's line of sight (e.g., in the vicinity of the haptic/optic junction).

In cases where the implantable ophthalmic device is an IOL, the IOL may have optical power provided by a transparent or translucent element with one or more curved surfaces or one or more graded refractive index profiles. Alternatively, the implantable ophthalmic device may be an IOO, which has little to no optical power except when actuated as described herein. In some illustrative devices, the optical element(s) and shape-memory members may provide a continuous range of focus between the fixed or static corrective powers of the ophthalmic lens.

As used herein, “ambient light” means light exterior to the eye. In some embodiments, ambient light refers more specifically to the light exterior to, but near or adjacent to the eye, e.g., light near the corneal surface. Ambient light can be characterized by variables such as the amount of light (e.g., intensity, radiance, luminance) and source of light (including both natural sources, e.g., sun and moon, as well as artificial sources such as incandescent, fluorescent, computer monitors, etc.).

As used herein, “accommodative response” refers to one or more physical or physiological events that enhance near vision. Natural accommodative responses, those that occur naturally in vivo, include, but are not limited to, ciliary muscle contraction, zonule movement, alteration of lens shape, iris sphincter contraction, pupil constriction, and convergence. The accommodative response can also be an artificial accommodative response, i.e., a response by an artificial optical component. Artificial accommodative responses include, but are not limited to, changing position, changing curvature, changing refractive index, or changing aperture size.

The accommodative response (also known as the accommodative loop) includes at least three involuntary ocular responses: (1) ciliary muscle contraction, (2) iris sphincter contraction (pupil constriction increases depth of focus), and (3) convergence (looking inward enables binocular fusion at the object plane for maximum binocular summation and best stereoscopic vision). Ciliary muscle contraction is related to accommodation per se: the changing optical power of the lens. Pupil constriction and convergence relate to pseudo-accommodation; they do not affect the optical power of the lens, but they nevertheless enhance near-object focusing. See, e.g., Bron A J, Vrensen G F J M, Koretz J, Maraini G, Harding 11.2000. The Aging Lens. Ophthalmologica 214:86-104, which is hereby incorporated herein by reference in its entirety.

As used herein, “accommodative impulse” refers to the intent or desire to focus on a near object. In a healthy, non-presbyopic eye, the accommodative impulse would be followed rapidly by the accommodative response. In a presbyopic eye, the accommodative impulse may be followed by a sub-optimal or absent accommodative response.

As used herein, “accommodative stimulus” is any detectable event or set of circumstances correlated to accommodative impulse or accommodative response. In the devices described herein, when an accommodative stimulus is detected by the sensor system, the sensor system preferably transmits a signal to an optical component, which in turn responds with an artificial accommodative response. Exemplary accommodative stimuli include, but are not limited to, physiological cues (such as pupil constriction and other natural accommodative responses) and environmental cues (such as ambient lighting conditions).

The following applications are incorporated herein by reference in their entireties:
U.S. Pat. No. 7,926,940 to Blum et al., issued Apr. 19, 2011, and entitled, “Advanced Electro-Active Optic Device”;
PCT/US2011/038597 filed May 31, 2011, and entitled, “Intermediate Vision Provided by an Aspheric IOL with an Embedded Dynamic Aperture”;
PCT/US2011/040896 filed Jun. 17, 2011, and entitled, “ASIC Design and Function”;
PCT/US2011/041764 filed Jun. 24, 2011, and entitled, “Use of Non Circular Optical Implants to Correct Aberrations in the Eye”;
U.S. Patent Application Publication No. 2010/0004741 filed Jul. 2, 2009, and entitled, “Sensor for Detecting Accommodative Trigger”;
U.S. Patent Application Publication No. 2011/0015733 filed Jul. 14, 2010, and entitled, “Folding Designs for Intraocular Lenses”;
PCT/US2011/050533 filed Sep. 6, 2011, and entitled, “Installation and Sealing of a Battery on a Thin Glass Wafer to Supply Power to an Intraocular Implant”;
PCT/US2011/051198 filed Sep. 12, 2011, and entitled, “Method and Apparatus for Detecting Accommodations”; and
PCT/US2011/060556 filed Nov. 14, 2011, and entitled “Adaptive Intraocular Lens.”

The use of flow diagrams is not meant to be limiting with respect to the order of operations performed. The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations.

However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.).

It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. An implantable ophthalmic device comprising:
an optical element;
a shape-memory member coupled to the optical element; and
a power supply operably coupled to the shape-memory member and configured to apply electromagnetic energy to at least a portion of the shape-memory member so as to cause the shape-memory member to change shape.

2. The implantable ophthalmic device of claim 1 wherein the optical element comprises a lens, a prism, an iris, or a spatial light modulator.

3. The implantable ophthalmic device of claim 1 wherein the optical element has an optical power of about +10 Diopters to about +36 Diopters.

4. The implantable ophthalmic device of claim 1 wherein the optical element is a first optical element and further comprising:
a second optical element in optical communication with the first optical element, and
wherein, when the shape-memory member undergoes a change in shape, the shape-memory member causes relative movement between the first optical element and the second optical element.

5. The implantable ophthalmic device of claim 4 wherein, when the shape-memory member undergoes a change in shape, the shape-memory member causes the first optical element to move by about 0.5 mm to about 1.5 mm relative to the second optical element

6. The implantable ophthalmic device of claim 4 wherein the first optical element has an optical power of about +0.5 Diopters to about +3.5 Diopters and the second optical element has an optical power of about +10 Diopters to about +35 Diopters.

7. The implantable ophthalmic device of claim 1 wherein the shape-memory member comprises a curved section that straightens when the power supply applies the electromagnetic energy to the shape-memory member.

8. The implantable ophthalmic device of claim 1 wherein, when implanted in an eye, the shape-memory member moves the optical element along an optical axis of the eye when subject to the electromagnetic energy.

9. The implantable ophthalmic device of claim 8 wherein movement of the optical element along the optical axis of the eye produces a change in optical power of about 0.1 Diopters to about 3.5 Diopters.

10. The implantable ophthalmic device of claim 1 wherein the shape-memory member comprises at least one of a shape-memory alloy and a shape-memory polymer.

11. The implantable ophthalmic device of claim 1 wherein the shape-memory member comprises a hydrogel.

12. The implantable ophthalmic device of claim 1 wherein the shape-memory member changes shape when heated to a temperature of about 35 degrees Celsius to about 45 degrees Celsius.

13. The implantable ophthalmic device of claim 1 wherein the shape-memory member changes shape when subjected to a power of about 1.5 microwatts to about 5.0 microwatts.

14. The implantable ophthalmic device of claim 1 wherein the shape-memory member is at least partially coated with an elastic polymeric material.

15. The implantable ophthalmic device of claim 14 polymeric material comprises at least one of cross-linked silicone, acrylic co-polymer, polyvinyledene difluoride, and polyimide.

16. The implantable ophthalmic device of claim 1 wherein the shape-memory member has a thickness of about 0.02 mm to about 0.10 mm.

17. The implantable ophthalmic device of claim 1 wherein the implantable ophthalmic device has a length of about 10.6 mm to about 13.0 mm.

18. The implantable ophthalmic device of claim 1 further comprising:
a first electrode configured to convey current from the power supply to a first portion of the shape-memory member;
a second electrode configured to convey current from the power supply to a second portion of the shape-memory member; and
a switch operably coupled to the first and second electrodes and configured to control application of the current to the first and second portions of the shape-memory member.

19. The implantable ophthalmic device of claim 18 wherein the first portion of the shape-memory member is configured to change shape independently of the second portion of the shape-memory member.

20. The implantable ophthalmic device of claim 18 wherein the switch is configured to apply current to the first portion of the shape-memory member independent of application of current to the second portion of the shape-memory member.

21. The implantable ophthalmic device of claim 18 further comprising:
a third electrode configured to convey current from the power supply to a third portion of the shape-memory member;

22. The implantable ophthalmic device of claim 1 wherein the power supply is configured to apply a current to the shape-memory member so as to heat the shape-memory member, thereby causing the shape-memory member to change shape.

23. The implantable ophthalmic device of claim 1 wherein the power supply is configured to apply a voltage to the shape-memory member so as to cause the shape-memory member to change shape.

24. The implantable ophthalmic device of claim 1 wherein the power supply comprises a rechargeable battery.

25. The implantable ophthalmic device of claim 1 further comprising:
a detector configured to provide a trigger signal indicative of the presence of an accommodative trigger; and
a processor operably coupled to the detector and the power supply and configured to actuate the power supply in response to the trigger signal.

26. A method of dynamic focusing:
applying electromagnetic energy to at least a portion of a shape-memory member coupled to an optical element so as to cause the shape-memory member to move the optical element.

27. The method of claim 26 wherein applying the electromagnetic energy comprises running a current through the at least a portion of the shape-memory member.

28. The method of claim 27 wherein running the current through the at least a portion of the shape-memory member causes the at least a portion of the shape-memory member to undergo a phase transition.

29. The method of claim 26 wherein applying the electromagnetic energy comprises applying a voltage across the at least a portion of the shape-memory member.

30. The method of claim 26 wherein applying the electromagnetic energy to the at least a portion of the shape-memory member causes the at least a portion of the shape-memory member to change shape.

31. The method of claim 30 wherein applying the electromagnetic energy to the at least a portion of the shape-memory member causes the at least a portion of the shape-memory member to straighten.

32. The method of claim 26 further comprising:
anchoring the optical element to a structure within the eye.

33. The method of claim 32 wherein applying the electromagnetic energy to the at least a portion of the shape-memory member causes the shape-memory member to move the optical element along an optical axis of the eye.

34. The method of claim 26 wherein the optical element is a first optical element and wherein applying the electromagnetic energy to the at least a portion of the shape-memory member causes the shape-memory member to move the first optical element with respect to the second optical element.

35. The method of claim 34 wherein the shape-memory member moves the first optical element by about 0.5 mm to about 1.5 mm relative to the second optical element

36. The method of claim 34 wherein movement of the optical element along the optical axis of the eye produces a change in optical power of about 0.1 Diopters to about 3.5 Diopters.

37. The method of claim 26 wherein the at least a portion of the shape-memory member is a first portion of the shape-memory member and further comprising:
applying electromagnetic energy to a second portion of the shape-memory member so as to cause the shape-memory member to move the optical element over an additional distance.

38. The method of claim 26 further comprising:
providing a trigger signal representing a presence of an accommodative trigger; and
applying the electromagnetic energy in response to the trigger signal.

39. An implantable ophthalmic device comprising:
a first optical element;
a second optical element in optical communication with the first optical element;
a shape-memory member coupled to at least one of the first and second optical elements;
a power supply operably coupled to the shape-memory member;
a detector configured to provide a trigger signal indicative of the presence of an accommodative trigger; and
a switch operably coupled to the detector and the power supply and configured to apply current from the power supply to at least a portion of the shape-memory member in response to the trigger signal so as to cause the shape-memory member to move the first optical element with respect to the second optical element.
